(19) United States
(12) Patent Application Publication
(10) Pub. No.: US 2026/0216723 A1
(43) Pub. Date: Jul. 30, 2026

(54) MICROFLUIDIC SORTING CHIP, DROPLET SCREENING METHOD, SYSTEM, DEVICE, AND STORAGE MEDIUM

(71) Applicant: BGI SHENZHEN, Guangdong (CN)

(72) Inventors: Xiangwen Yan, Guangdong (CN); Hui Zhang, Guangdong (CN); Mengzhe Shen, Guangdong (CN); Haoran Xu, Guangdong (CN); Xiang Li, Guangdong (CN); Shiyue Zhou, Guangdong (CN); Peiyi Wang, Guangdong (CN); Yue Zheng, Guangdong (CN); Wenwei Zhang, Guangdong (CN); Yuliang Dong, Guangdong (CN)

(73) Assignee: BGI SHENZHEN, Guangdong (CN)

(21) Appl. No.: 19/142,168

(22) PCT Filed: Dec. 30, 2022
(86) PCT No.: PCT/CN2022/143858

§ 371 Date: Jun. 20, 2025

(51) Int. Cl. B01L 3/00; G01N 21/64

(52) U.S. Cl. CPC B01L 3/502715; G01N 21/6428; B01L 2200/10; B01L 2300/0645

(57) ABSTRACT

The present application provides a microfluidic sorting chip, a droplet screening method, a system, a device, and a storage medium. Wherein the droplet screening method comprises: according to a preset frequency, acquiring at least two types of electrical signals of a droplet to be detected; determining within the electrical signals a target electrical signal which satisfies an intensity threshold; when the signal width of the target electrical signal satisfies a signal width threshold, extracting features from target electrical signal data, judging whether intra-channel features of the target electrical signal satisfy a first preset condition, and judging whether inter-channel features of the target electrical signal satisfy a second preset condition; and if so, then judging that the droplet to be detected is a target droplet.

### TECHNICAL FIELD

The present disclosure relates to the field of microfluidics technology, for example, to a microfluidic sorting chip, a droplet screening method, a system, a device, and a storage medium.

### BACKGROUND

Enzymes are a class of macromolecular biological catalysts with significant applications across various fields such as industry and medicine. Different usage scenarios demand distinct enzyme performance characteristics, including thermal stability, stability in organic solvents, catalytic efficiency, substrate specificity, etc. Therefore, screening for high-performance enzymes is critically important. In the enzyme screening process based on microdroplet technology, single cells and reaction substrates are encapsulated within microdroplets for reactions. A key step involves signal amplification, acquisition, and data processing of droplet signals at specific bands, making decisions and sorting based on the characteristics of signals in each channel. Simultaneously, the enzyme screening method based on microdroplet technology can be applied to scenarios such as cell line development and monoclonal antibody development.

Existing technical means include ELISA screening technology, spectrophotometer screening technology, gel electrophoresis screening technology, flow cytometer screening technology and microfluidic single-channel screening technology, etc. The above technical means have the shortcoming of being unable to provide diversified screening solutions.

### SUMMARY

The technical problem to be solved by the present disclosure is to provide a microfluidic sorting chip, a droplet screening method, a system, a device, and a storage medium in order to overcome the shortcoming in the prior art that fails to provide diversified screening solutions.

The present disclosure solves the above technical problem through the following technical solutions:

In a first aspect, the present disclosure provides a droplet screening method, the method comprising:
sampling at least two types of electrical signals of a droplet to be detected at a preset frequency, each type of electrical signal of the droplet to be detected being acquired in different analog-to-digital conversion signal channels;
determining a target electrical signal satisfying an intensity threshold among the electrical signals;
extracting features from data of the target electrical signal when a signal width of the target electrical signal satisfies a signal width threshold, judging whether intra-channel features of the target electrical signal satisfy a first preset condition, and judging whether inter-channel features of the target electrical signal satisfy a second preset condition;
if so, identifying the droplet to be detected as a target droplet;
wherein the intra-channel features are features of the droplet to be detected which correspond to features within each analog-to-digital conversion signal channel, and the inter-channel features are features of the droplet to be detected which correspond to features between each analog-to-digital conversion signal channel.

Optionally, the signal width is acquired by calculating based on the number of the target electrical signals satisfying the intensity threshold and sampling frequency.

Optionally, the step of judging whether intra-channel features of the target electrical signal satisfy a first preset condition comprises:
acquiring an average intensity and a peak intensity of the target electrical signal satisfying the signal width threshold;
judging that the first preset condition is satisfied when the intra-channel features of the target electrical signal satisfying the signal width threshold satisfy a first ratio threshold;
wherein the intra-channel feature is a ratio of the average intensity to the peak intensity of the target electrical signal satisfying the signal width threshold.

Optionally, the inter-channel features comprise a first inter-channel feature and a second inter-channel feature, and the step of judging whether inter-channel features of the target electrical signal satisfy a second preset condition comprises:
acquiring the target electrical signal satisfying the first preset condition and acquiring the peak intensity;
judging that the second preset condition is satisfied when the first inter-channel feature of the target electrical signal satisfying the first preset condition satisfies a second ratio threshold;
wherein the inter-channel feature is a ratio between the peak intensities of the target electrical signals satisfying the first preset condition between respective analog-to-digital conversion signal channels corresponding to the droplet to be detected;
and/or,
acquiring the target electrical signal satisfying the first preset condition and acquiring the average intensity;
judging that the second preset condition is satisfied when the second inter-channel feature of the target electrical signal satisfying the first preset condition satisfies a third ratio threshold;
wherein the inter-channel feature is a ratio between the average intensities of the target electrical signals satisfying the first preset condition between respective analog-to-digital conversion signal channels corresponding to the droplet to be detected.

Optionally, the step of sampling at least two types of electrical signals of a droplet to be detected at a preset frequency comprises:
acquiring an optical signal of the droplet to be detected via a photomultiplier tube, and sampling the optical signal at a preset frequency and acquiring at least two types of the electrical signals of the droplet to be detected;
wherein the optical signal comprises at least one type of a scattered light signal, a fluorescence signal, and an absorbed light signal.

Optionally, the step of determining a target electrical signal satisfying an intensity threshold among the electrical signals further comprises:
collecting the electrical signals into a data packet when the at least two types of electrical signals of the droplet to be detected sampled at the preset frequency satisfy the intensity threshold for the first time;
until the electrical signals fall below the intensity threshold;
wherein the data packet collects all electrical signals from the first time when at least two types of electrical signals of the droplet to be detected sampled at the preset frequency satisfy the intensity threshold to the last time the intensity threshold is satisfied.

In a second aspect, the present disclosure provides a microfluidic sorting chip. The microfluidic sorting chip comprises a sorting component and at least two branch channels. When the droplet to be detected is identified as the target droplet by the droplet screening method described in any one of the foregoing, the sorting component is configured to receive a deflection instruction and sort the droplet to be detected into a corresponding branch channel.

In a third aspect, the present disclosure provides a droplet screening system, the system comprising:
an acquisition module, configured to sample at least two types of electrical signals of a droplet to be detected at a preset frequency, each type of electrical signal of the droplet to be detected being acquired in different analog-to-digital conversion signal channels;
a determination module, configured to determine a target electrical signal satisfying an intensity threshold among the electrical signals;
a judgment module, configured to extract features from data of the target electrical signal when a signal width of the target electrical signal satisfies a signal width threshold, judge whether intra-channel features of the target electrical signal satisfy a first preset condition, and judge whether inter-channel features of the target electrical signal satisfy a second preset condition;
if so, identify the droplet to be detected as a target droplet;
wherein the intra-channel features are features of the droplet to be detected which correspond to features within each analog-to-digital conversion signal channel, and the inter-channel features are features of the droplet to be detected which correspond to features between each analog-to-digital conversion signal channel.

In a fourth aspect, the present disclosure provides an electronic device comprising a memory, a processor, and a computer program stored in the memory and used to run on the processor, wherein the computer program, when executed by the processor, implements the droplet screening method described in any one of the foregoing.

In a fifth aspect, the present disclosure provides a computer-readable storage medium having a computer program stored thereon, wherein the computer program, when executed by a processor, implements the droplet screening method described in any one of the foregoing.

The positive progressive effect of the present disclosure is that: by sampling the at least two types of electrical signals of the droplet to be detected at the preset frequency and determining the target electrical signal satisfying the intensity threshold among the electrical signals, features are extracted from data of the target electrical signal when the signal width of the target electrical signal satisfies the signal width threshold, and whether the droplet to be detected is the target droplet is identified by judging whether the intra-channel features of the target electrical signal satisfy the first preset condition and judging whether the inter-channel features of the target electrical signal satisfy the second preset condition. A variety of screening schemes are provided for screening the droplets to be detected, thereby improving the flexibility and accuracy of screening the droplets to be detected.

### BRIEF DESCRIPTION OF THE DRAWINGS

FIG. 1 is a flow chart of a droplet screening method provided in Example 1 of the present disclosure.

FIG. 2 is a schematic diagram of a structure for performing droplet screening through a microfluidic sorting chip provided in Example 1 of the present disclosure.

FIG. 3 is a partial flow chart of a droplet screening method provided in Example 1 of the present disclosure.

FIG. 4 is a partial flow chart of another droplet screening method provided in Example 1 of the present disclosure.

FIG. 5 is a partial flow chart of yet another droplet screening method provided in Example 1 of the present disclosure.

FIG. 6 is a flow chart of droplet screening using two signals from channel 1 and channel 3 provided in Example 1 of the present disclosure.

FIG. 7 is a schematic diagram of modules of a droplet screening system provided in an example of the present disclosure.

FIG. 8 is a schematic structural diagram of an electronic device provided in Example 4 of the present disclosure.

### DETAILED DESCRIPTION OF THE PREFERRED EMBODIMENT

The present disclosure is further described below through examples, but it is not limited to the scope of these examples.

### Example 1

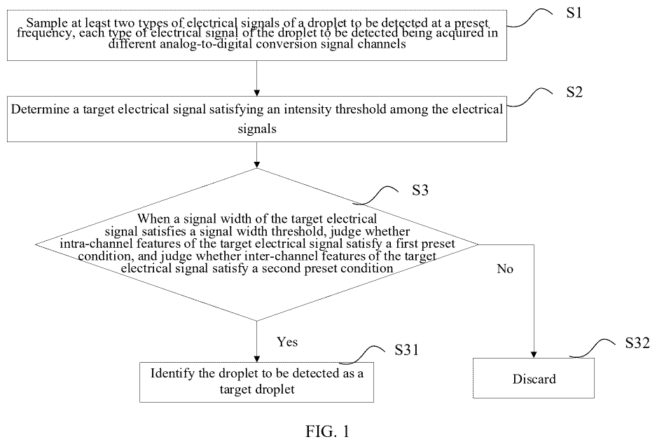

Referring to FIG. 1, a droplet screening method comprises:
S1: sampling at least two types of electrical signals of a droplet to be detected at a preset frequency, each type of electrical signal of the droplet to be detected being acquired in different analog-to-digital conversion signal channels.

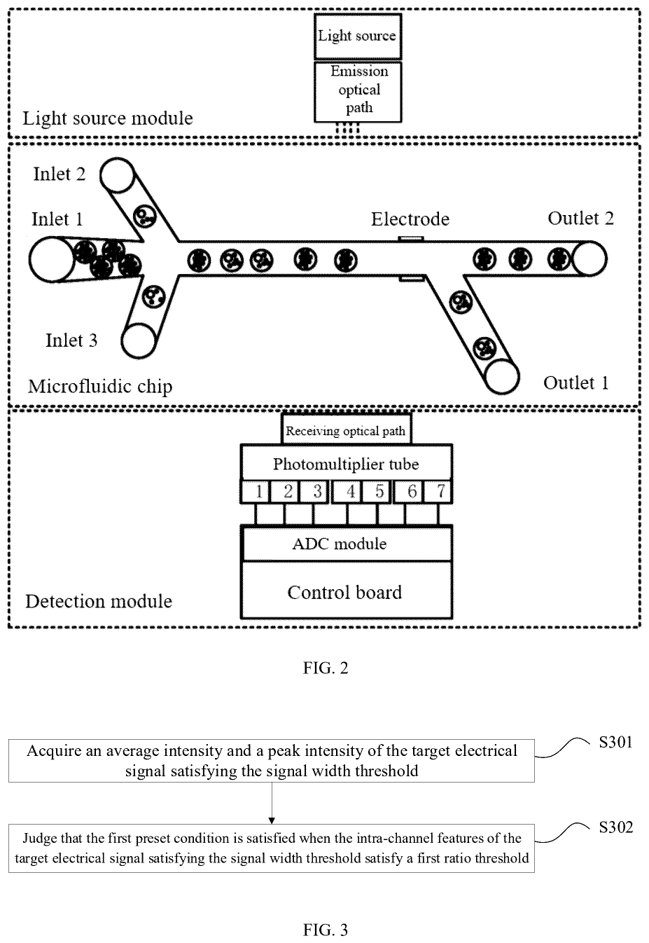

Herein, a microfluidic sorting chip may be used when performing droplet screening. The droplets to be detected are arranged in a single row in the flow channel of the microfluidic sorting chip. When their spacing remains stable during passage, a light source passes through an emission optical path and is focused on the detection point of the microfluidic sorting chip. The light source and the emission optical path are light source modules. Referring to FIG. 2, a receiving optical path installed at the other end uses a multi-channel photomultiplier tube (e.g., a 7-channel photomultiplier tube) to convert the optical signals of fluorescence, scattered light, and absorbed light into voltage signals. Multi-channel photomultiplier tubes are independently connected to an ADC module consisting of multiple ADCs (analog-to-digital converters). Those skilled in the art should understand that the ADC module is composed of several analog-to-digital converters. The ADC module, the resolution of the ADC module, and the ADC sampling frequency are determined by the actual situation. At least two types of electrical signals of the droplet to be detected are sampled at the preset frequency, and each type of electrical signal of the droplet to be detected is acquired in different analog-to-digital conversion signal channels. For example, a droplet to be detected is sampled at a frequency of 50 KHZ, and electrical signals corresponding to the scattered light and the fluorescence of the droplet to be detected are sampled, that is, the electrical signals corresponding to the scattered light and fluorescence are acquired in two analog-to-digital conversion signal channels. Among them, an ADC module composed of 3 ADCs is relatively common. If the resolution of the ADC is 12, the reference voltage is 3.3 V, and the ADC reading is V_{adc}, then the numerical conversion formula for the voltage level corresponding to the droplet is:

$$X = \frac{3.3 \times 1000 \times {V}_{adc}}{4096} ,$$

with a unit of millivolt.

Background signal filtering can be accomplished through a control board connected to the ADC module, while characteristic values of multiple signals (different optical signals or parameters of the same optical signal in different bands), including but not limited to peak, average, and signal width, are sampled. Intra-channel as well as inter-channel signal calculations are performed to screen the droplets to be detected.

S2: determining a target electrical signal satisfying an intensity threshold among the electrical signals.

Among the at least two types of electrical signals of the droplet to be detected sampled at the preset frequency across different analog-to-digital conversion signal channels, the target electrical signal is the electrical signal satisfying the intensity threshold from a starting point (when acquiring the electrical signals that simultaneously satisfy the intensity threshold for the first time in at least two analog-to-digital conversion signal channels) to an end point (when an electrical signal in one analog-to-digital conversion signal channel fails to satisfy the intensity threshold). For example, the electrical signals corresponding to scattered light and fluorescence of the droplet to be detected are sampled, and the electrical signals satisfying the intensity threshold in the two analog-to-digital conversion signal channels are acquired as the target electrical signals. The intensity threshold of each analog-to-digital conversion signal channel is determined according to the biological characteristics of the droplet to be detected. The voltage level is usually 500 mV (millivolts) to 1100 mV (millivolts). The target electrical signal is the electrical signal satisfying the intensity threshold from a starting point (when acquiring the electrical signals that simultaneously satisfy the intensity threshold for the first time in two analog-to-digital conversion signal channels) to an end point (when an electrical signal in one analog-to-digital conversion signal channel fails to satisfy the intensity threshold).

S3: extracting features from the target electrical signal when a signal width of the target electrical signal satisfies a signal width threshold, judging whether intra-channel features of the target electrical signal satisfy a first preset condition, and judging whether inter-channel features of the target electrical signal satisfy a second preset condition; in the case where the judgment result is yes, executing step S31.

S31: identifying the droplet to be detected as a target droplet.

When the signal widths of the target electrical signals acquired in different analog-to-digital conversion signal channels all satisfy the signal width threshold, it is judged whether the intra-channel features of the target electrical signal satisfy the first preset condition and whether the inter-channel features of the target electrical signal satisfy the second preset condition. If so, the droplet to be detected is identified as the target droplet. If not, S32 of discarding is executed.

Herein, the intra-channel features are features of the droplet to be detected which correspond to features within each analog-to-digital conversion signal channel, and the inter-channel features are features of the droplet to be detected which correspond to features between each analog-to-digital conversion signal channel.

In this example, by sampling the at least two types of electrical signals of the droplet to be detected at the preset frequency and determining the target electrical signal satisfying the intensity threshold among the electrical signals, features are extracted from data of the target electrical signal when the signal width of the target electrical signal satisfies the signal width threshold, and whether the droplet to be detected is the target droplet is identified by judging whether the intra-channel features of the target electrical signal satisfy the first preset condition and judging whether the inter-channel features of the target electrical signal satisfy the second preset condition. A variety of screening schemes are provided for screening the droplets to be detected, thereby improving the flexibility and accuracy of screening the droplets to be detected.

Optionally, the signal width is acquired by calculating based on the number of the target electrical signals satisfying the intensity threshold and sampling frequency.

The formula for calculating signal width is:

$${S}_{w} = ( N - 1 ) * {T}_{sample} ;$$

where S_{w} is the signal width, N is the number of target electrical signals satisfying the intensity threshold for one droplet to be detected within one analog-to-digital conversion signal channel, and Tₛₐₘₚₗₑ is the sampling interval time for a droplet to be detected.

In this example, by calculating the signal width, a variety of screening schemes are provided for screening the droplets to be detected, thereby improving the flexibility and accuracy of screening the droplets to be detected.

Optionally, referring to FIG. 3, the step of judging whether intra-channel features of the target electrical signal satisfy a first preset condition in step S3 comprises:
S301: acquiring an average intensity and a peak intensity of the target electrical signal satisfying the signal width threshold.

Among them, the average intensity and peak intensity of the voltage level of the target electrical signal satisfying the signal width threshold in different analog-to-digital conversion signal channels are acquired. For example, a droplet to be detected has N target electrical signal voltage levels that meet the intensity threshold in an analog-to-digital conversion signal channel, which are numbered as, Xᵢ, Xᵢ₊₁ . . . . X_{N-1}, X_{N}, respectively. The peak intensity (Vₘₐₓ) of the target electrical signal is the peak voltage level among them, and the calculation formula for the average intensity of the voltage level of the target electrical signal is:

$${V}_{avg} = \frac{{\sum }_{i = 1}^{N} {X}_{i}}{N} .$$

S302: judging that the first preset condition is satisfied when the intra-channel features of the target electrical signal satisfying the signal width threshold satisfy a first ratio threshold, wherein the intra-channel feature is a ratio of the average intensity to the peak intensity of the target electrical signal satisfying the signal width threshold.

The formula for calculating the intra-channel feature (Ri ma) is:

$${R}_{i\_ma} = \frac{{V}_{max}}{{V}_{avg}} \times 100 \% ;$$

When the intra-channel features satisfy the first ratio threshold, then the first preset condition is satisfied, and the first ratio threshold is set according to the biological characteristics of the droplet to be detected.

In this example, by calculating the intra-channel features and judging whether the intra-channel features satisfy the first preset condition, a variety of screening schemes are provided for screening the droplets to be detected, thereby improving the flexibility and accuracy of screening the droplets to be detected.

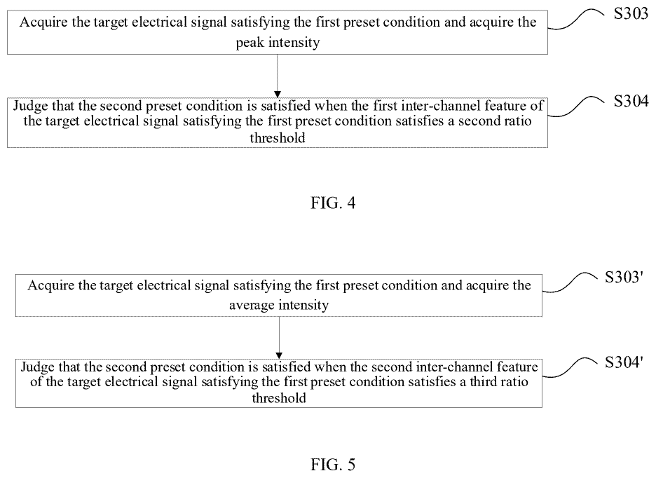

Optionally, the inter-channel features comprise a first inter-channel feature and a second inter-channel feature. Referring to FIG. 4, the step of judging whether inter-channel features of the target electrical signal satisfy a second preset condition comprises:
S303: acquiring the target electrical signal satisfying the first preset condition and acquiring the peak intensity;
S304: judging that the second preset condition is satisfied when the first inter-channel feature of the target electrical signal satisfying the first preset condition satisfies a second ratio threshold; wherein the inter-channel feature is a ratio between the peak intensities of the target electrical signals satisfying the first preset condition between respective analog-to-digital conversion signal channels corresponding to the droplet to be detected.

For example, the peak intensities of voltage levels of the target electrical signals satisfying the first preset condition within two analog-to-digital conversion signal channels for a droplet to be detected are obtained, respectively, and the first inter-channel feature (R_{b_mm}) is calculated. The calculation formula is:

$${R}_{{b}_{-} m m} = \frac{{V}_{max 1}}{{V}_{max 2}} \times 100 \% ;$$

where Vₘₐₓ₁ is the peak intensity of the voltage level of the target electrical signal satisfying the first preset condition within one analog-to-digital conversion signal channel, and Vₘₐₓ₂ is the peak intensity of the voltage level of the target electrical signal satisfying the first preset condition in the other analog-to-digital conversion signal channel. When the first inter-channel feature satisfies the second ratio threshold, then the second preset condition is satisfied.

And/or, referring to FIG. 5,
S303′: acquiring the target electrical signal satisfying the first preset condition and acquiring the average intensity;
S304′: judging that the second preset condition is satisfied when the second inter-channel feature of the target electrical signal satisfying the first preset condition satisfies a third ratio threshold; wherein the inter-channel feature is a ratio between the average intensities of the target electrical signals satisfying the first preset condition between respective analog-to-digital conversion signal channels corresponding to the droplet to be detected.

For example, the average intensities of voltage levels of the target electrical signals satisfying the first preset condition within two analog-to-digital conversion signal channels for a droplet to be detected are obtained, respectively, and the second inter-channel feature (R_{b_aa}) is calculated. The calculation formula is:

$${R}_{b\_aa} = \frac{{V}_{avg 1}}{{V}_{avg 2}} \times 100 \% ;$$

where V_{avg1} is the average intensity of the voltage level of the target electrical signal satisfying the first preset condition within one analog-to-digital conversion signal channel, and V_{avg2} is the average intensity of the voltage level of the target electrical signal satisfying the first preset condition in the other analog-to-digital conversion signal channel. When the second inter-channel feature satisfies the third ratio threshold, then the second preset condition is satisfied.

In this example, by calculating the first inter-channel feature and the second inter-channel feature and judging whether the inter-channel features satisfy the second preset condition, a variety of screening schemes are provided for screening the droplets to be detected, thereby improving the flexibility and accuracy of screening the droplets to be detected.

Optionally, the step of sampling at least two types of electrical signals of a droplet to be detected at a preset frequency comprises:
acquiring an optical signal of the droplet to be detected via a photomultiplier tube, and sampling the optical signal at a preset frequency and acquiring at least two types of the electrical signals of the droplet to be detected; wherein the optical signal comprises at least one type of a scattered light signal, a fluorescence signal, and an absorbed light signal.

Among them, at least one type of the fluorescence signal, the scattered light signal, and the absorbed light signal is selected for screening according to the different biological characteristics of the microdroplets to be detected. For example, the fluorescence signal and scattered light of a droplet to be detected correspond to two analog-to-digital conversion signal channels respectively; or, two bands of the fluorescence signal of a droplet to be detected correspond to two analog-to-digital conversion signal channels. Different optical signals and the bands corresponding to the optical signals may be selected according to the biological characteristics of the droplet to be detected.

In this example, by selecting fluorescence signals, scattered light signals, and absorbed light signals as well as selecting different bands according to the biological characteristics of the droplet to be detected, a variety of screening schemes are provided for screening the droplets to be detected, thereby improving the flexibility and accuracy of screening the droplets to be detected.

Optionally, step S2 further comprises:
collecting the electrical signals into a data packet when the at least two types of electrical signals of the droplet to be detected sampled at the preset frequency satisfy the intensity threshold for the first time, until the electrical signals fall below the intensity threshold. The data packet collects all electrical signals from the first time when at least two types of electrical signals of the droplet to be detected sampled at the preset frequency satisfy the intensity threshold to the last time the intensity threshold is satisfied.

Among the at least two types of electrical signals of the droplet to be detected sampled at the preset frequency across different analog-to-digital conversion signal channels, the electrical signals satisfying the voltage level intensity threshold from a starting point (when acquiring the electrical signals that simultaneously satisfy the voltage level intensity threshold for the first time in at least two analog-to-digital conversion signal channels) to an end point (when an electrical signal in one analog-to-digital conversion signal channel fails to satisfy the voltage level intensity threshold) are collected into the data packet. When the voltage level intensity threshold is satisfied, the data is updated to the data packet, and the electrical signal voltage level, peak voltage level, and count are updated at the same time. Those skilled in the art should understand that, in addition to the voltage level, other parameters may also be used for measurement, and different parameters may be selected according to actual conditions. In addition to data packets, data may also be stored in data tables or data links. Data stored in data packets, data tables or data links are analyzed, and electrical signals are analyzed.

In this example, by collecting data packets to provide data support for the subsequent process of screening droplets to be detected, a variety of screening schemes are provided for screening the droplets to be detected, thereby improving the flexibility and accuracy of screening the droplets to be detected.

Optionally, when the droplet to be detected is judged to be a positive droplet, the excitation electrode generates a high-voltage level to deflect the droplet, thereby completing the screening. Herein, the width of the pulse level is selected based on the liquid flow rate and the distance between the detection point and the electrode, with a selectable range of 500 to 4000 (microseconds).

Optionally, the communication protocol is customized according to the actual screening scheme, and operations such as querying the number of screening droplets and starting and stopping the screening program are performed via the host computer.

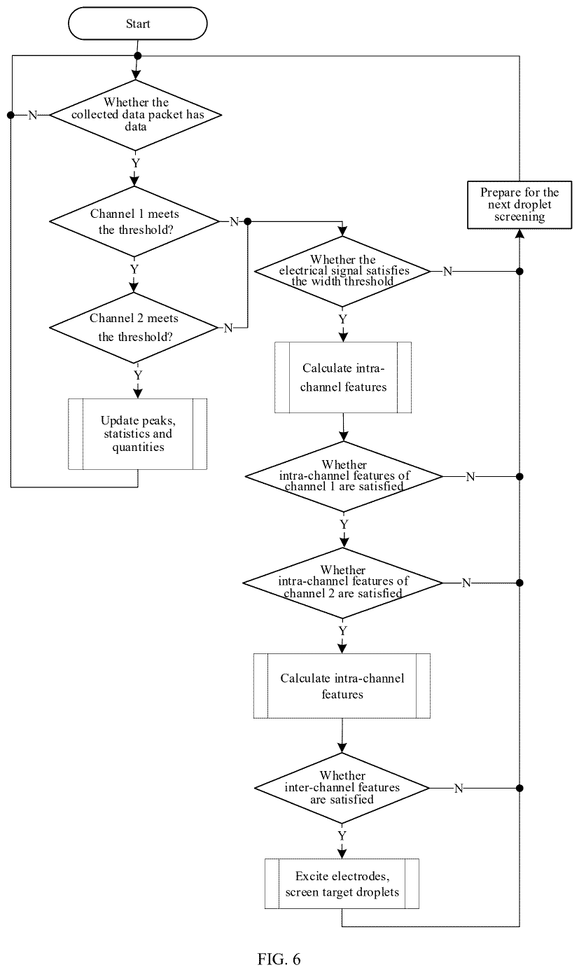

In a practical example, referring to FIG. 6, two signals from channel 1 and channel 3 are used for droplet screening:
1) Load the aqueous phase and droplet-generation oil into a syringe. Connect the syringe to a tube, then insert the tube into the corresponding inlet of the microfluidic chip. Open the syringe to allow droplets to enter the microfluidic sorting chip.
2) Adjust the light intensity and position of the light source so that the light spot is focused on the detection point; monitor the voltage level of the droplet signal through an oscilloscope and control the signal spike voltages between 2V (volt-ampere) and 3V (volt-ampere) to ensure that the light intensity and position are suitable for the subsequent screening process.
3) Adjust the syringe flow rate and capture the interval between two droplet signals through an oscilloscope so that the interval between the two droplet signals is about 4 milliseconds, that is, the flow rate is controlled at 250 droplets/second.
4) The sampling frequency has been set to 100 KHz in the software, that is, the sampling interval is Tₛₐₘₚₗₑ=10 microseconds.
5) Set parameters.

Channel 1 parameters: voltage level intensity threshold V_{thr_1}=850 mV, voltage level average intensity V_{avg_1}=1250 mV, voltage level peak intensity V_{max_1}=1600 mV, signal width threshold S_{w_1}=120;

Channel 3 parameters: voltage level intensity threshold V_{thr_3}=750 mV, voltage level average intensity V_{avg_3}=1100 mV, voltage level peak intensity V_{max_3}=1300 mV, signal width threshold S_{w_3}=120.

First inter-channel feature:

$${R}_{b\_mm} = \frac{{V}_{max\_ 1}}{{V}_{max \_ 3}} \times 100 \% \geq 112.$$

6) Turn on the timer and start screening.

7) Intra-channel judgment: when the electrical signal voltage level in channel 1≥V_{thr_1} and the electrical signal voltage level in channel 3≥V_{thr_3}: if this condition is satisfied for the first time, it is recorded as the signal starting point and the data is stored. When the electrical signal voltage level in channel 1<V_{thr_1} and the electrical signal voltage level in channel 3<V_{thr_3}: calculate and judge whether the signal width in channel 1≥S_{w_1} and the signal width in channel 3≥S_{w_3}. If both conditions are satisfied, proceed to step 8) to judge the intra-channel feature conditions; otherwise, clear the cache and proceed to the next droplet screening.

8) Inter-channel judgment:
Channel 1 judgment: average voltage level of electrical signal≥V_{avg_1}, peak voltage level of electrical signal≥V_{max_1};
Channel 3 judgment: average voltage level of electrical signal≥V_{avg_3}, peak voltage level of electrical signal≥V_{max_3}. If the two channel feature conditions are satisfied, proceed to step 9) to judge the inter-channel feature conditions; otherwise clear the cache and proceed to the next droplet screening.

9) Calculate the voltage level peak intensity ratio≥R_{b_mm}. If the condition is satisfied, excite the electrode to deflect the microdroplet and screen a droplet; otherwise, clear the cache and proceed to the next droplet screening.

10) Repeat steps 7), 8), and 9) to achieve automatic screening of microdroplets. During the screening process, the host computer may query the number of screened droplets every 1 second according to the communication protocol, which is convenient for statistics and analysis of the distribution of droplets.

### Example 2

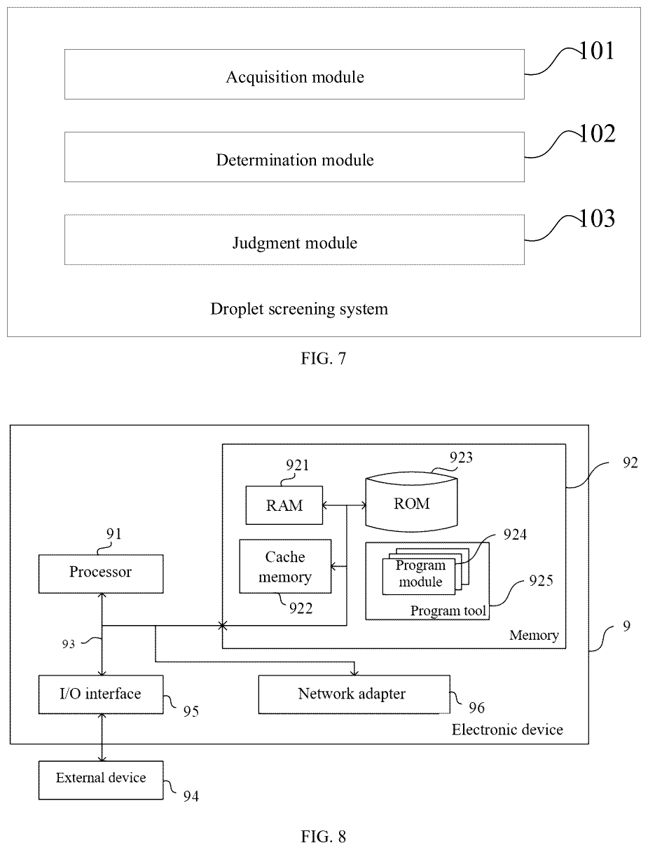

Referring to FIG. 7, a droplet screening system comprises:
an acquisition module 101, configured to sample at least two types of electrical signals of a droplet to be detected at a preset frequency, each type of electrical signal of the droplet to be detected being acquired in different analog-to-digital conversion signal channels.

Herein, a microfluidic sorting chip may be used when performing droplet screening. The droplets to be detected are arranged in a single row in the flow channel of the microfluidic sorting chip. When their spacing remains stable during passage, a light source passes through an emission optical path and is focused on the detection point of the microfluidic sorting chip. Referring to FIG. 2, a receiving optical path installed at the other end uses a multi-channel photomultiplier tube (e.g., a 7-channel photomultiplier tube) to convert the optical signals of fluorescence, scattered light, and absorbed light into voltage signals. Multi-channel photomultiplier tubes are independently connected to an ADC module consisting of multiple ADCs (analog-to-digital converters). Those skilled in the art should understand that the ADC module is composed of several analog-to-digital converters. The ADC module, the resolution of the ADC module, and the ADC sampling frequency are determined by the actual situation. At least two types of electrical signals of the droplet to be detected are sampled at the preset frequency, and each type of electrical signal of the droplet to be detected is acquired in different analog-to-digital conversion signal channels. For example, a droplet to be detected is sampled at a frequency of 50 KHZ, and electrical signals corresponding to the scattered light and the fluorescence of the droplet to be detected are sampled, that is, the electrical signals corresponding to the scattered light and fluorescence are acquired in two analog-to-digital conversion signal channels. Among them, an ADC module composed of 3 ADCs is relatively common. If the resolution of the ADC is 12, the reference voltage is 3.3 V, and the ADC reading is V_{adc}, then the numerical conversion formula for the voltage level corresponding to the droplet is:

$$X = \frac{3.3 \times 1000 \times {V}_{adc}}{4096} ,$$

with a unit of millivolt.

A determination module 102, configured to determine a target electrical signal satisfying an intensity threshold among the electrical signals.

Among the at least two types of electrical signals of the droplet to be detected sampled at the preset frequency across different analog-to-digital conversion signal channels, the target electrical signal is the electrical signal satisfying the intensity threshold from a starting point (when acquiring the electrical signals that simultaneously satisfy the intensity threshold for the first time in at least two analog-to-digital conversion signal channels) to an end point (when an electrical signal in one analog-to-digital conversion signal channel fails to satisfy the intensity threshold). For example, the electrical signals corresponding to scattered light and fluorescence of the droplet to be detected are sampled, and the electrical signals satisfying the intensity threshold in the two analog-to-digital conversion signal channels are acquired as the target electrical signals. The intensity threshold of each analog-to-digital conversion signal channel is determined according to the biological characteristics of the droplet to be detected. The voltage level is usually 500 mV (millivolts) to 1100 mV (millivolts). The target electrical signal is the electrical signal satisfying the intensity threshold from a starting point (when acquiring the electrical signals that simultaneously satisfy the intensity threshold for the first time in two analog-to-digital conversion signal channels) to an end point (when an electrical signal in one analog-to-digital conversion signal channel fails to satisfy the intensity threshold).

A judgment module 103, configured to extract features from data of the target electrical signal when a signal width of the target electrical signal satisfies a signal width threshold, judge whether intra-channel features of the target electrical signal satisfy a first preset condition, and judge whether inter-channel features of the target electrical signal satisfy a second preset condition.

If so, the droplet to be detected is identified as a target droplet.

Herein, the intra-channel features are features of the droplet to be detected which correspond to features within each analog-to-digital conversion signal channel, and the inter-channel features are features of the droplet to be detected which correspond to features between each analog-to-digital conversion signal channel.

When the signal widths of the target electrical signals acquired in different analog-to-digital conversion signal channels all satisfy the signal width threshold, it is judged whether the intra-channel features of the target electrical signal satisfy the first preset condition and whether the inter-channel features of the target electrical signal satisfy the second preset condition. If so, the droplet to be detected is identified as the target droplet.

Herein, the intra-channel features are features of the droplet to be detected which correspond to features within each analog-to-digital conversion signal channel, and the inter-channel features are features of the droplet to be detected which correspond to features between each analog-to-digital conversion signal channel.

In this example, by sampling the at least two types of electrical signals of the droplet to be detected at the preset frequency and determining the target electrical signal satisfying the intensity threshold among the electrical signals, features are extracted from data of the target electrical signal when the signal width of the target electrical signal satisfies the signal width threshold, and whether the droplet to be detected is the target droplet is identified by judging whether the intra-channel features of the target electrical signal satisfy the first preset condition and judging whether the inter-channel features of the target electrical signal satisfy the second preset condition. A variety of screening schemes are provided for screening the droplets to be detected, thereby improving the flexibility and accuracy of screening the droplets to be detected.

Optionally, the signal width is acquired by calculating based on the number of the target electrical signals satisfying the intensity threshold and sampling frequency.

Optionally, when judging whether intra-channel features of the target electrical signal satisfy the first preset condition, the judgment module is specifically configured to:
acquire an average intensity and a peak intensity of the target electrical signal satisfying the signal width threshold;
judge that the first preset condition is satisfied when the intra-channel features of the target electrical signal satisfying the signal width threshold satisfy a first ratio threshold;
wherein the intra-channel feature is a ratio of the average intensity to the peak intensity of the target electrical signal satisfying the signal width threshold.

Optionally, the inter-channel features comprise a first inter-channel feature and a second inter-channel feature, and when judging whether the inter-channel features of the target electrical signal satisfy the second preset condition, the judgment module is specifically configured to:
acquire the target electrical signal satisfying the first preset condition and acquire the peak intensity;
judge that the second preset condition is satisfied when the first inter-channel feature of the target electrical signal satisfying the first preset condition satisfies a second ratio threshold;
wherein the inter-channel feature is a ratio between the peak intensities of the target electrical signals satisfying the first preset condition between respective analog-to-digital conversion signal channels corresponding to the droplet to be detected;
and/or,
acquire the target electrical signal satisfying the first preset condition and acquire the average intensity;
judge that the second preset condition is satisfied when the second inter-channel feature of the target electrical signal satisfying the first preset condition satisfies a third ratio threshold;
wherein the inter-channel feature is a ratio between the average intensities of the target electrical signals satisfying the first preset condition between respective analog-to-digital conversion signal channels corresponding to the droplet to be detected.

Optionally, the acquisition module is specifically configured to:
acquire an optical signal of the droplet to be detected via a photomultiplier tube, and sample the optical signal at a preset frequency and acquire at least two types of the electrical signals of the droplet to be detected;
wherein the optical signal comprises at least one type of a scattered light signal, a fluorescence signal, and an absorbed light signal.

Optionally, the determination module is specifically configured to:
collect the electrical signals into a data packet when the at least two types of electrical signals of the droplet to be detected sampled at the preset frequency satisfy the intensity threshold for the first time;
until the electrical signals fall below the intensity threshold;
wherein the data packet collects all electrical signals from the first time when at least two types of electrical signals of the droplet to be detected sampled at the preset frequency satisfy the intensity threshold to the last time the intensity threshold is satisfied.

As for the system example, since it basically corresponds to the method example, the relevant parts may refer to the partial description of the method example. The system example described above is merely illustrative, wherein the units described as separate components may or may not be physically separated, and the components displayed as units may or may not be physical units, that is, they may be located in one place or distributed over multiple network units. Some or all of the modules may be selected according to actual needs to achieve the purpose of the solution of the present disclosure. A person skilled in the art can understand and implement the present disclosure without any creative work.

### Example 3

The present disclosure provides a microfluidic sorting chip. The microfluidic sorting chip comprises a sorting component and at least two branch channels. When the droplet to be detected is identified as the target droplet by the droplet screening method of the above Example 1, the sorting component is configured to receive a deflection instruction and sort the droplet to be detected into a corresponding branch channel.

Herein, referring to FIG. 2, the aqueous phase and the oil phase enter the microfluidic chip from inlet 1, inlet 2, and inlet 3 to form microdroplets. When the droplet to be detected is identified as the target droplet, the sorting component receives a deflection instruction and deflects the droplet through an excitation electrode. Finally, the target droplets and waste droplets flow out from the branch channels and from outlet 1 and outlet 2.

In this example, the use of the microfluidic sorting chip makes the amount of reagent used in the microdroplets very small, thereby greatly reducing the cost.

### Example 4

This example provides an electronic device, which may be implemented in the form of a computing device (e.g., a server device), including a memory, a processor, and a computer program stored in the memory and executable on the processor, wherein the computer program, when executed by the processor, may implement the droplet screening method provided in Example 1.

FIG. 8 shows a schematic diagram of the hardware structure of this example. As shown in FIG. 8, the electronic device 9 specifically includes:
at least one processor 91, at least one memory 92, and a bus 93 for connecting different system components (including the processor 91 and the memory 92), wherein:
the bus 93 includes a data bus, an address bus, and a control bus.

The memory 92 includes a volatile memory, such as a random-access memory (RAM) 921 and/or a cache memory 922, and may further include a read-only memory (ROM) 923.

The memory 92 further includes a program/utility 925 having a set of (at least one) program modules 924, such program modules 924 including, but not limited to, an operating system, one or more application programs, other program modules, and program data. Each of these examples or some combination thereof may include a component for implementing a network environment.

The processor 91 runs the computer program stored in the memory 92, thereby executing various functional applications and data processing, such as the droplet screening method provided in Example 1 of the present disclosure.

The electronic device 9 may further communicate with one or more external devices 94. Such communication is established through an input/output (I/O) interface 95. Moreover, the electronic device 9 may also communicate with one or more networks (e.g., a local area network (LAN), a wide area network (WAN), and/or a public network, such as the Internet) via a network adapter 96. The network adapter 96 communicates with other modules of the electronic device 9 via the bus 93. It should be understood that although not shown in figures, other hardware and/or software modules can be used in combination with the electronic device 9, including, but not limited to, a microcode, a device driver, a redundant processor, an external disk drive array, a RAID (Redundant Array of Independent Disks) system, a tape drive, a data backup storage system, etc.

It should be noted that although several units/modules or sub-units/modules of the electronic device are mentioned in the detailed description above, such a division is merely exemplary and not mandatory. In practice, according to the embodiments of the present application, the features and functions of two or more units/modules described above may be embodied in a single unit/module. Conversely, the features and functions of a single unit/module described above may be further divided to be embodied by multiple units/modules.

### Example 5

This example provides a computer-readable storage medium having a computer program stored thereon, wherein the program, when executed by a processor, implements the droplet screening method provided in Example 1.

Herein, the readable storage medium may employ devices more specifically, including, but not limited to, a portable disk, a hard disk, a random-access memory, a read-only memory, an erasable programmable read-only memory, an optical storage device, a magnetic storage device, or any suitable combination of the above.

In a possible embodiment, the present disclosure may also be implemented in the form of a program product comprising a program code which, when the program product is run on a terminal device, causes the terminal device to perform the droplet screening method provided in Example 1.

Herein, the program code for executing the present disclosure may be written in any combination of one or more programming languages, and may be executed entirely on the user device, partially on the user device, as a standalone software package, partially on the user device and partially on a remote device, or entirely on a remote device.

Although specific embodiments of the present disclosure have been described above, it should be understood by those skilled in the art that this is merely illustrative, and the scope of protection of the present disclosure is defined by the appended claims. Those skilled in the art may make various changes or modifications to these embodiments without departing from the principles and essence of the present disclosure, but such changes and modifications fall within the scope of protection of the present disclosure.

1. A droplet screening method, wherein the method comprises:
sampling at least two types of electrical signals of a droplet to be detected at a preset frequency, each type of electrical signal of the droplet to be detected being acquired in different analog-to-digital conversion signal channels;
determining a target electrical signal satisfying an intensity threshold among the electrical signals;
extracting features from data of the target electrical signal when a signal width of the target electrical signal satisfies a signal width threshold, judging whether intra-channel features of the target electrical signal satisfy a first preset condition, and judging whether inter-channel features of the target electrical signal satisfy a second preset condition;
if so, identifying the droplet to be detected as a target droplet;
wherein the intra-channel features are features of the droplet to be detected which correspond to features within each analog-to-digital conversion signal channel, and the inter-channel features are features of the droplet to be detected which correspond to features between each analog-to-digital conversion signal channel.

2. The droplet screening method according to claim 1, wherein the signal width is acquired by calculating based on the number of the target electrical signals satisfying the intensity threshold and sampling frequency.

3. The droplet screening method according to claim 1, wherein the step of judging whether intra-channel features of the target electrical signal satisfy a first preset condition comprises:
acquiring an average intensity and a peak intensity of the target electrical signal satisfying the signal width threshold;
judging that the first preset condition is satisfied when the intra-channel features of the target electrical signal satisfying the signal width threshold satisfy a first ratio threshold;
wherein the intra-channel feature is a ratio of the average intensity to the peak intensity of the target electrical signal satisfying the signal width threshold.

4. The droplet screening method according to claim 3, wherein the inter-channel features comprise a first inter-channel feature and a second inter-channel feature, and the step of judging whether inter-channel features of the target electrical signal satisfy a second preset condition comprises:
acquiring the target electrical signal satisfying the first preset condition and acquiring the peak intensity;
judging that the second preset condition is satisfied when the first inter-channel feature of the target electrical signal satisfying the first preset condition satisfies a second ratio threshold;
wherein the inter-channel feature is a ratio between the peak intensities of the target electrical signals satisfying the first preset condition between respective analog-to-digital conversion signal channels corresponding to the droplet to be detected;
and/or,
acquiring the target electrical signal satisfying the first preset condition and acquiring the average intensity;
judging that the second preset condition is satisfied when the second inter-channel feature of the target electrical signal satisfying the first preset condition satisfies a third ratio threshold;
wherein the inter-channel feature is a ratio between the average intensities of the target electrical signals satisfying the first preset condition between respective analog-to-digital conversion signal channels corresponding to the droplet to be detected.

5. The droplet screening method according to claim 1, wherein the step of sampling at least two types of electrical signals of a droplet to be detected at a preset frequency comprises:
acquiring an optical signal of the droplet to be detected via a photomultiplier tube, and sampling the optical signal at a preset frequency and acquiring at least two types of the electrical signals of the droplet to be detected;
wherein the optical signal comprises at least one type of a scattered light signal, a fluorescence signal, and an absorbed light signal.

6. The droplet screening method according to claim 1, wherein the step of determining a target electrical signal satisfying an intensity threshold among the electrical signals further comprises:
collecting the electrical signals into a data packet when the at least two types of electrical signals of the droplet to be detected sampled at the preset frequency satisfy the intensity threshold for the first time;
until the electrical signals fall below the intensity threshold;
wherein the data packet collects all electrical signals from the first time when at least two types of electrical signals of the droplet to be detected sampled at the preset frequency satisfy the intensity threshold to the last time the intensity threshold is satisfied.

7. A microfluidic sorting chip, wherein the microfluidic sorting chip comprises a sorting component and at least two branch channels, wherein when the droplet to be detected is identified as the target droplet by the droplet screening method according to claim 1, the sorting component is configured to receive a deflection instruction and sort the droplet to be detected into a corresponding branch channel.

8. A droplet screening system, wherein the system comprises:
an acquisition module, configured to sample at least two types of electrical signals of a droplet to be detected at a preset frequency, each type of electrical signal of the droplet to be detected being acquired in different analog-to-digital conversion signal channels;
a determination module, configured to determine a target electrical signal satisfying an intensity threshold among the electrical signals;
a judgment module, configured to extract features from data of the target electrical signal when a signal width of the target electrical signal satisfies a signal width threshold, judge whether intra-channel features of the target electrical signal satisfy a first preset condition, and judge whether inter-channel features of the target electrical signal satisfy a second preset condition;
if so, identify the droplet to be detected as a target droplet;
wherein the intra-channel features are features of the droplet to be detected which correspond to features within each analog-to-digital conversion signal channel, and the inter-channel features are features of the droplet to be detected which correspond to features between each analog-to-digital conversion signal channel.

9. An electronic device comprising a memory, a processor, and a computer program stored in the memory and used to run on the processor, wherein the computer program, when executed by the processor, implements the droplet screening method according to claim 1.

10. A computer-readable storage medium having a computer program stored thereon, wherein the computer program, when executed by a processor, implements the droplet screening method according to claim 1.

11. A microfluidic sorting chip, wherein the microfluidic sorting chip comprises a sorting component and at least two branch channels, wherein when the droplet to be detected is identified as the target droplet by the droplet screening method according to claim 2, the sorting component is configured to receive a deflection instruction and sort the droplet to be detected into a corresponding branch channel.

12. A microfluidic sorting chip, wherein the microfluidic sorting chip comprises a sorting component and at least two branch channels, wherein when the droplet to be detected is identified as the target droplet by the droplet screening method according to claim 3, the sorting component is configured to receive a deflection instruction and sort the droplet to be detected into a corresponding branch channel.

13. A microfluidic sorting chip, wherein the microfluidic sorting chip comprises a sorting component and at least two branch channels, wherein when the droplet to be detected is identified as the target droplet by the droplet screening method according to claim 4, the sorting component is configured to receive a deflection instruction and sort the droplet to be detected into a corresponding branch channel.

14. A microfluidic sorting chip, wherein the microfluidic sorting chip comprises a sorting component and at least two branch channels, wherein when the droplet to be detected is identified as the target droplet by the droplet screening method according to claim 5, the sorting component is configured to receive a deflection instruction and sort the droplet to be detected into a corresponding branch channel.

15. A microfluidic sorting chip, wherein the microfluidic sorting chip comprises a sorting component and at least two branch channels, wherein when the droplet to be detected is identified as the target droplet by the droplet screening method according to claim 6, the sorting component is configured to receive a deflection instruction and sort the droplet to be detected into a corresponding branch channel.
